(19) United States
(12) Patent Application Publication
(10) Pub. No.: US 2024/0189012 A1
(43) Pub. Date: Jun. 13, 2024

(54) PLASMA GAS GENERATOR

(71) Applicant: Thomas J. SHEPERAK, SWANTON, OH (US)

(72) Inventor: Thomas J. SHEPERAK, SWANTON, OH (US)

(21) Appl. No.: 18/223,924

(22) Filed: Jul. 19, 2023

Related U.S. Application Data
(63) Continuation of application No. PCT/US22/13488, filed on Jan. 24, 2022.
(63) Continuation-in-part of application No. 16/272,416, filed on Feb. 11, 2019.
(63) Continuation-in-part of application No. 16/003,550, filed on Jun. 8, 2018, now abandoned.
Application No. 16/003,550 is a continuation of application No. 14/117,119, filed on Jun. 27, 2014, now Pat. No. 9,993,282.
(60) Provisional application No. 63/140,852, filed on Jan. 23, 2021.
(60) Provisional application No. 63/141,416, filed on Jan. 25, 2021.
(60) Provisional application No. 62/629,929, filed on Feb. 13, 2018.
(60) Provisional application No. 61/485,747, filed on May 13, 2011.

(51) Int. Cl. A61B 18/04; A61L 2/00; A61N 1/44; A61N 5/10; G21K 1/093; H05H 1/46

(52) U.S. Cl. CPC A61B 18/042; A61L 2/007; A61N 1/44; A61N 5/10; G21K 1/093; H05H 1/46; H05H 1/4645; A61N 2005/1089; H05H 1/466; H05H 2240/20; H05H 2242/26; H05H 2245/34

(57) ABSTRACT

A method of generating an output gas, comprising plasmatizing an input gas with RF power propagating from a tip of an electrode to form an annular plasma sheath constrained by a tube with said RF power propagating within said annular plasma sheath; and forming said output gas as said annular plasma sheath propagates away from said tip of said electrode.

### REFERENCE TO RELATED APPLICATION

This application is a continuation-in-part of U.S. application Ser. No. 16/272,416, filed Feb. 11, 2019, which claims the benefit of U.S. Provisional Application. 62/629,929, filed Feb. 13, 2018, and this application is a continuation of PCT/US22/13488, filed Jan. 24, 2022, which claims the benefit of U.S. Provisional Application No. 63/140,852, filed Jan. 23, 2021, and U.S. Provisional Application No. 63/141,416, filed Jan. 25, 2021, and this application is a continuation-in-part of U.S. application Ser. No. 16/003,550, filed Jun. 8, 2018, which is a continuation of U.S. application Ser. No. 14/117,119, filed Jun. 27, 2014, which is a national stage application of International Application No. PCT/US12/37249, filed May 10, 2012, which claims the benefit of U.S. Provisional Application. 61/485,747, filed May 13, 2011, each of which are hereby incorporated by reference in their entirety.

### FIELD OF INVENTION

This invention relates generally to producing useful gases with a plasma directed electron beam, and, more specifically, to a method and apparatus for producing nitric oxides using a plasma directed electron beam.

### BACKGROUND

Nitric oxides (NxOx) are known as antimicrobial and sterilization agents, and have many applications in the life sciences. However, due to their often toxic nature and short shelf life, their availability is limited. What is needed is a means of generating NxOx, as needed, to avoid problems with their limited shelf-life, and avoid/reduce the need to inventory/stock toxic gases. The present invention fulfills this need among others.

### SUMMARY

The following presents a simplified summary of the invention in order to provide a basic understanding of some aspects of the invention. This summary is not an extensive overview of the invention. It is not intended to identify key/critical elements of the invention or to delineate the scope of the invention. Its sole purpose is to present some concepts of the invention in a simplified form as a prelude to the more detailed description that is presented later.

The Coaxial Energy Delivery System described in U.S. Pat. No. 9,993,282 (herein incorporated by reference in its entirety) utilizes the insulating properties of plasma as a waveguide to confine and direct a coherent electromagnetic wave of electrical energy at Radio Frequency (RF). In other words, the system uses plasma to provide a path for the delivery of the electromagnetic wave to a target material. This can be described as plasma “sheath” on the outside and beam on the inside, the plasma and the electromagnetic wave travelling coaxially from the point of emission to a target material and, as such, define a Plasma Directed Electron Beam (PDEB).

By manipulating the magnetic component of an electromagnetic wave, the electrons from a power source are accelerated to the point wherein the electrons, as a compressed wave, are spontaneously emitted from a metallic conductor surrounded by a tubular or columnar flow of a gas, such as helium, argon, and other Nobel gases, and/or atmospheric air. The electrons are accelerated to sufficient speed to energize the tubular or columnar flow of gas to the plasma state which, using the radio frequency insulating properties of plasma, confines and directs the electromagnetic wave, as a visible beam, coaxially, to a target material some distance away from the emitter. Neither the emission of the wave nor coupling of the wave to a target material requires a physical ground path return.

The relationship between the coaxial flow of the electromagnetic wave and the tubular or columnar flow of the gas energized to plasma state is symbiotic. The flow of gas depends upon the electromagnetic wave as its energy source to reach the plasma state, and the electromagnetic wave depends upon the plasma to guide its travel to reach a target. Therefore, the electromagnetic wave energy creates its own conduit or waveguide as it propagates along its axis, coaxially with the plasma column.

The outside portion of the plasma column serves as an insulator to confine and compress the electromagnetic wave at radio frequency, but, paradoxically, on the inside, the plasma column serves as a super conductor to direct the electromagnetic wave to a target material. Therefore, unlike a conventional radio station broadcast antenna in which the RF energy emanates in an ever expanding sphere, the PDEB surrounds the RF energy with a column of gas and creates a waveguide that prevents the radio frequency from expanding as a sphere. The compressed radio frequency can be directed with high energy density from the RF emitter to point to a target material which can be a liquid, a solid or a gas.

The electromagnetic wave delivered to a target in this coaxial system is at such a frequency so as to resonate either at the primary or at a harmonic frequency thereof, thereby elevating the electrons of such material to a higher energy state which, among other things, results in the emission of photons. This causes the target materials to more easily and efficiently melt, vaporize, dissociate, or otherwise be modified, depending upon the desired outcome. This coaxial energy delivery system provides a reliable means to electro-chemically manipulate and alter matter.

Applicant has discovered that the PDEB not only melts, vaporizes, or dissociates target materials, but also dissociates gas molecules used to form the plasma in a selective and predictable way. Without being tied to a particular theory, Applicant believes that the PDEB provides a consistent and predictable radial power density through the plasma sheath as the PDEB propagates from the electrode. In other words, because the plasma sheath has a thin annular profile, the electron beam within the beam is able to uniformly excite the beam radially. This means that, not only is the power density consistent through the plasma sheath proximate to the electrode tip such that the disassociation/ionization of atoms in the plasma is assured, but also, as the RF power wave propagates away from the electrode tip and loses energy, the reduced power density the plasma sheath is also consistent, thereby selectively allowing atoms to combine. For example, if air is used as the plasma gas, the PDEB may be energized to disassociate the diatomic molecules N2 and O2 found in air to their monatomic units—i.e., N and O. Then, as the plasma sheath propagates away from the end of the electrode, and the radial power density in the plasma sheath drops, atoms—e.g., nitrogen and oxygen—will combine in predictable ways based on the consistent radio power density. More specifically, in this particular example, because nitrogen is in abundance and oxygen scarce, and because the eV for nitrogen is considerably higher than that of oxygen, as the radial power density across the plasma sheath drops, the abundant nitrogen will be more likely to combine with the scarce oxygen to form NO rather than other combinations—e.g. nitrogen dioxide (NO2).

Therefore, one important feature of the PDEB is its ability to consistently energize the plasma beam radially. This means that elements are ionized consistently near the tip of the electrode radially across the plasma beam, and then, as the power of the electron beam diminishes as the beam propagates way form the electrode tip, the ionized elements combined with other elements in a predictable way due to the consistent radial energy across the plasma beam.

Conversely, conventional plasma generating devices utilizing an arc, inductive coupling, capacitive discharge and the like, focus only on creating a plasma and rely only on the plasma, itself, to do work, utilizing the free electrons of the plasma only. In such systems the number of free electrons is inconsistent. In each of these systems, the flow of gas as the feedstock for the plasma, is introduced to flow perpendicular to the energy source. For example, in an arc plasma system, utilizing two electrodes, the gas flows across the arc and the energy within the arc is imparted to the gas, providing energy to create a plasma state in the gas. The energy density contained in the plasma is reduced proportionately to the distance a cross section of the plasma travels away from the arc. Additionally, an arc is a violent occurrence, electrically, causing degradation and wear on the electrode materials. This necessitates periodic replacement of the electrodes along with related maintenance.

It should be understood that the system and method disclosed herein has various applications in addition to producing nitric oxides. Other applications include at least the following, just to name a few:
· The production of free hydrogen and nanoscale free carbon particles by dissociating, for example, methane (CH4) and other hydrocarbon materials.
Vaporizing solid, liquid, and gaseous materials for deposition of coating materials.
Modifying the surfaces of materials by raising surface energy to improve bonding of coatings on such materials and improve bonding together of those materials.
Reversing the charge on carbon particles to cause the homogenous separation of carbon nanomaterials for uniform disbursement of such particles onto substrate materials.
Reversing the charge on powdered coal to provide more surface area on the coal particles for complete combustion of coal.
Providing the economical use of electric power for melting and vaporizing materials as exemplified by sublimating tungsten at 3422º C (6192º F) using only 400 Watts of power through this Plasma Directed Electron Beam.
Destruction and desiccation of prokaryotic organisms (Pathogens) for the sterilization of surfaces, including mammalian cells at surgical sites, without harm to the mammalian cells.
The acceleration of wound healing by stimulating blood flow to the wound site and by stimulating fibroblasts which release collagen, among other things, to accelerate such wound healing.
The acceleration of tendon, ligament, and other soft tissue healing through the topical introduction of nitric oxide gas to the wound area.
The destruction of pathogenic organisms in the human oral cavity that cause periodontal and other oral diseases, without harm to the mammalian cells.
Heating atmospheric air for the purpose of drying grains.
Purifying water by destroying pathogens and neutralizing the toxicity of hazardous materials in water.
Destroying pathogens in and reducing the volume of human and animal sewage in sewage treatment.
Destroying medical waste.
Pyrolyzing municipal waste to recover methane.

According, in one embodiment, the invention comprises a method of generating an output gas, comprising: (a) plasmatizing an input gas with RF power propagating from a tip of an electrode to form an annular plasma sheath constrained by a tube with the RF power propagating within the annular plasma sheath; and (b) forming the output gas as the annular plasma sheath propagates away from the tip of the electrode.

In another embodiment, the invention comprises gas generator system, comprising: (a) a radio frequency (RF) power source; (b) at least one reactor having a first end and a second end, the reactor comprising at least, (i) a gas input at the first end for receiving an input gas from a gas source; (ii) an elongated tube having an axis; (iii) an electrode disposed at the first end with at least a portion of the electrode axially disposed within the tube, the electrode operatively connected to the RF power source, the electrode having a tip configured to emit the RF power such the RF power propagates axially along the tube, the electrode defining a channel for receiving the input gas from the gas input and for exhausting the input gas into the tube such that the input gas flows axially and laminarly along the tube; and (iv) a gas output in fluid communication with the tube to receive an output gas from the tube; wherein the RF power from the electrode and the flow of the input gas along the tube are sufficient for the RF power to plasmatize the input gas to form an annular plasma sheath constrained by the tube with the RF power propagating within the sheath; wherein the output gas forms from the annular plasma sheath as the annular plasma sheath propagates away from the tip of the electrode.

### BRIEF DESCRIPTION OF DRAWINGS

FIG. 1 shows one embodiment of the system of the present invention for generating nitric oxide.

FIG. 2 shows a top view of the embodiment of FIG. 1.

FIG. 3 shows a cross-sectional view of a plurality of reactors of the system of FIG. 1 with an outlet manifold.

FIG. 4 shows the art of connection between one of the reactors and the RF coil.

FIG. 5 shows an embodiment in which a plurality of igniters 501 are shown for igniting the annular plasma sheath.

FIG. 6 shows the embodiment of FIG. 5 with the covers over the first and second ends of the reactors.

FIG. 7 shows a top view of the reactors.

### DETAILED DESCRIPTION

In the following paragraphs, the present invention will be described in detail by way of example with reference to the attached drawings. Throughout this description, the preferred embodiment and examples shown should be considered as exemplars, rather than as limitations on the present invention. As used herein, the “present invention” refers to any one of the embodiments of the invention described herein, and any equivalents. Furthermore, reference to various feature(s) of the “present invention” throughout this document does not mean that all claimed embodiments or methods must include the referenced feature(s).

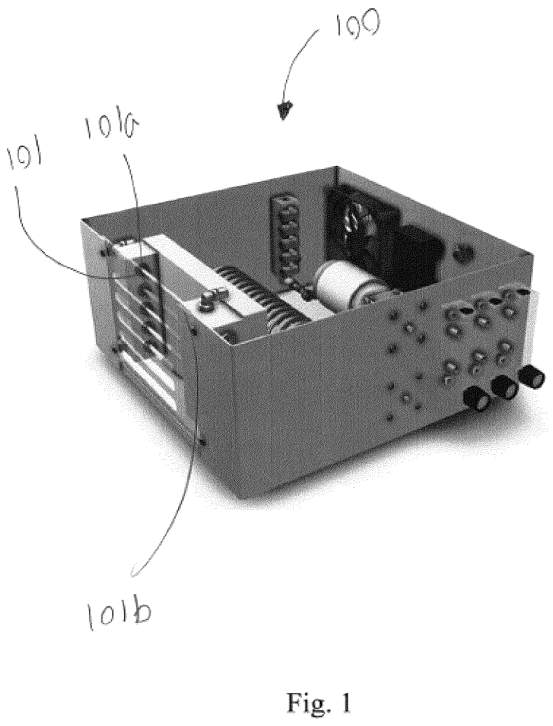

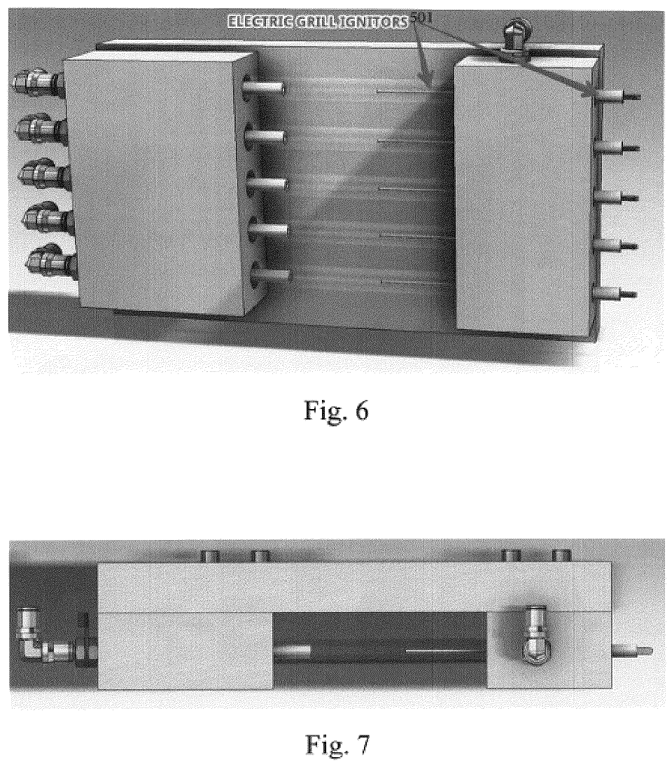

Referring to FIGS. 1-7, one embodiment of the gas producing system 100 is shown. The system may be used for ionizing elements of one gas or a combination of gases, and then facilitating their controlled combination to form a new gas. In one embodiment, the system produces nitric oxides (NxOx) and other gases. In one embodiment, the output gases including one or more of the following: NO, NO2, NO3, N2O, N2O2, N2O3, N2O4, N2O5, N4O, N4O6, HNO3, H2O2 and O3.

The system 100 is shown in perspective view. In this embodiment, system 100 has a plurality of reactors 101, each having a first end 101a and the second end 101b. It should be understood that, in these figures, various hoses and conductors connecting inputs and outputs are eliminated for simplicity/clarity.

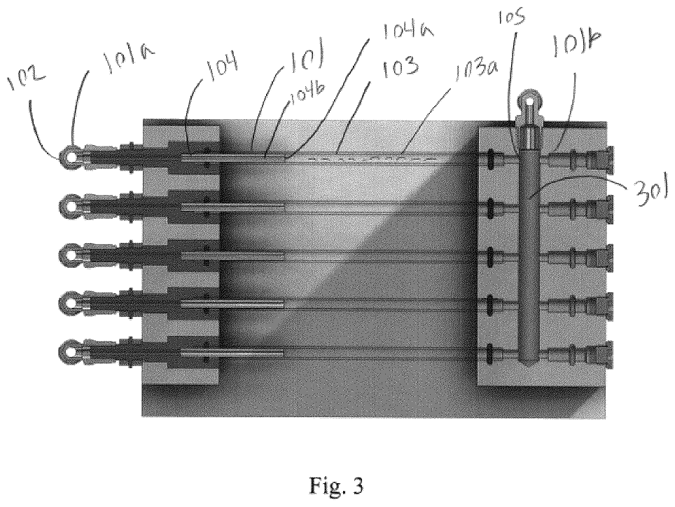

Referring to FIG. 3, the reactors 101 of system 100 are shown in cross-section. In one embodiment, each reactor comprises at least a gas input 102 at the first end for receiving the input gas from the gas source, an elongated tube 103 having an axis 103a, an electrode 104. The electrode is operatively connected to the RF power source, and is disposed at the first end with at least a portion of the electrode axially disposed within the tube. The electrode has a tip 104a configured to emit the RF power such the RF power propagates axially along the tube. The electrode defines a channel 104b for receiving the input gas from the gas input and for exhausting the input gas into the tube such that the input gas flows axially and laminarly along the tube. A gas output 105 is in fluid communication with the tube to receive an output gas from the tube.

The input gas comprises an ionization gas, and, optionally reactant gases, depending on the desired output gas. For example, in one embodiment, NO is produced, and air is used not only as the ionization gas, but also as the primary reactant gas. In one embodiment, an additional reactant gases are added to the input gas to improve yields and/or to vary the output gas/gases. (In this respect, it should be understood that the input gas and output gas may be single component gases or may be a mixture of gases.) For example, in one embodiment, a noble gas, such as helium and argon, can be added to an oxygen stream to ensure ionization produces essentially 100% NO as opposed to NOx. Other reactant gases may include, for example, oxygen, nitrogen, carbon dioxide, hydrogen, argon, methane, helium, krypton, neon, and other gases including water vapor, just to name a few.

In one embodiment, the input gas, including the ionization gas and additional reactant gases, if any, is drawn into the system using a vacuum pump, and the gas flow is controlled by a flowmeter. In another embodiment, rather than a vacuum pump, a positive pressure pump can be used to essentially pump the gas to the system. Still other means of introducing the gas into the system will be obvious to those of skill in the art in light of this disclosure. Moreover, the flow of the input gas can be optimized by one of skill in the art in light of this disclosure without undue experimentation. For example, in one embodiment, in which the input gas is air, and the output gas is nitric oxide (NO), a flow rate for the air was 30 ft.³ per hour at 32 psi has been shown to provide suitable results.

In the embodiment of FIG. 3, there are five reactors. In this particular embodiment, the gas output of each of the five reactors is connected to a common output manifold 301. The nitric oxide or other gas produced by the system of the present invention can be used as it exits the reaction chamber, or it can be stored/compressed for later use.

The electrode can be configured in different ways and may comprise different materials. In one embodiment, the electrode is tungsten, although other materials may be used, such as silver or iridium. The tube may also be configured in different ways and may comprise different materials. In one embodiment, the tube comprises a chemically non-reactive, heat-resistant material, such as glass, quartz, fused silica and mullite, although other more durable/tougher materials like heat-resistant polymers may be preferred.

In one embodiment, the system 100 is configured to produce nitric oxide. In such an embodiment, Applicant has found that suitable results have been achieved with a 6″ long quartz tube, having an OD of 0.25″, and an ID of 0.17″, and with a 2″ long tungsten electrode, having an OD of 0.17″ and an ID of 0.05″. It should be understood that these dimensions are provided just for illustration, and that those of skill in the art will be able to optimize the tube in the electrode for a given application in light of this disclosure.

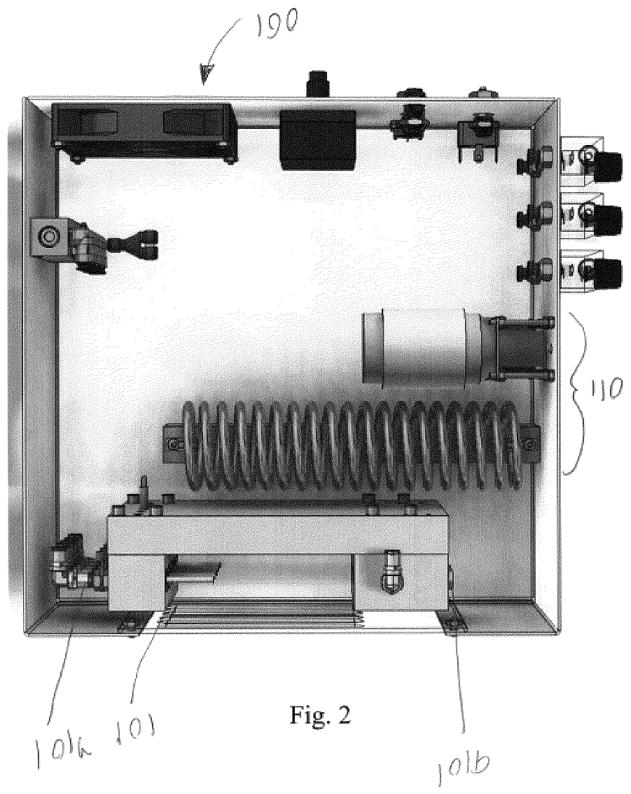

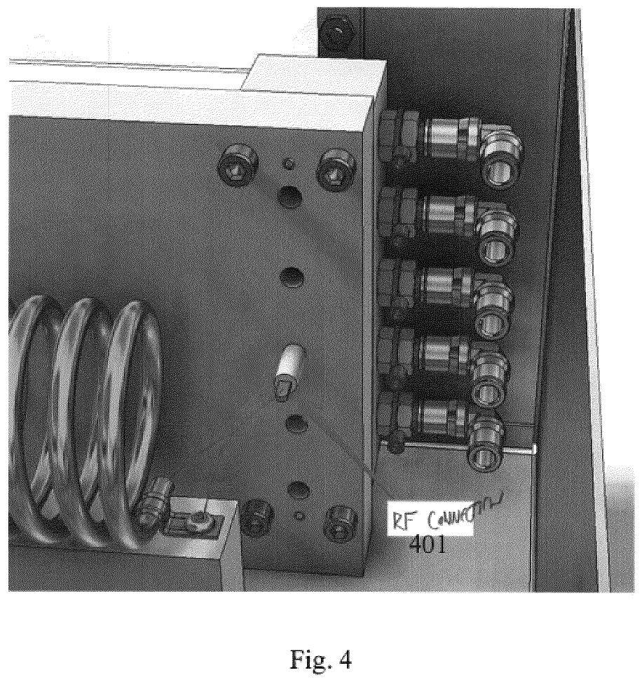

FIG. 2 shows a top perspective of the system 100. In this view, the RF tuning system 110 is shown, which, in this particular embodiment, comprises a coil and capacitor(s) for increasing the RF power (e.g. voltage) received from an RF power generator (not shown). The RF power generator is described in Applicant's U.S. Pat. No. 9,993,282 mentioned above. The RF power is connected to the electrode at RF connector 401 as shown in FIG. 4.

Generally, the RF power used to ionize the gas should be higher than the electron volt (eV) of gas molecules. For example, Air is roughly 1 part Oxygen (O2) and 4 parts Nitrogen (N2). It takes 9.76 eV to dissociate N2 and 5.11 eV to dissociate O2. This technology provides a consistent energy level to dissociate the N2 which is higher than the energy level required to dissociate O2. In one embodiment, in the production of NO the RF power is preferably greater than 30W, or greater than 50W, or greater than 75W, or greater than 100W or greater than 120W or greater than 150W or greater than 175W. In one particular embodiment, the RF power coupled to the electrode is around 130W. In one embodiment, the RF power has a frequency of 13.56 mHz, although other frequencies can be used.

The primary frequency, being radio frequency as the preferred embodiment herein, can be used as a carrier wave onto which an additional frequency or additional frequencies can be added. These additional frequencies would be the primary or a harmonic to the resonating frequency of the target material. Moreover, the coaxial energy delivery system can operate using alternative wave forms from a power generator, including but not limited to square wave, sinusoidal wave, triangular wave, saw tooth wave, pulsed direct current, direct current, or other wave forms.

It should be understood that one of skill in the art, in light of this disclosure, can optimize the RF frequency and power. For example, in one embodiment, in which air is the input gas and nitric oxide is the output gas, suitable results have been obtained using an RF power of 130 W, at a frequency of 13.56 mHz.

As described above, the RF power from the electrode and the flow of the input gas along the tube are sufficient for the RF power to plasmatize the input gas to form an annular plasma sheath constrained by the tube with the RF power propagating within the sheath. As the energy of the RF power diminishes as it propagates from the electrode tip, the ionized elements of the plasma begin to combine. In one embodiment, the output gas forms from the annular plasma sheath as the annular plasma sheath propagates away from the tip of the electrode.

The coaxial energy delivery system is scalable and is not limited to a single point of energy emission. Given the high potential energy available, as is represented on a sinusoidal wave representing the energy field, the high potential can be time-shared over a large area. Given the linear propagation of the electromagnetic wave, the linear path represented by the sinusoidal wave has a measurable length along which additional energy emitters can be fabricated to provide gas flow needed to form a columnar plasma at a point of electrical discharge, each of which emitter will cause a visible beam, each being comprised of plasma on the outside and beam (electromagnetic wave) on the inside. The means to create each additional beams can be as simple as drilling numerous holes in an electrically conductive tube, sealed at one end and with fittings provided at the other end to allow for the introduction of a flow of gas to be used to form the plasma waveguide. The conductive tube can be a length of several feet or there can be several tubes of shorter lengths with such holes and which tubes can be placed such as to form any geometric pattern to suit coverage of the coaxial energy delivery systems for an intended use.

In one embodiment, the electrode has an orifice axially defined at the tip. Applicant has found that the laminar exhausting of gas from an axially disposed orifice in the electrode facilitates plasmatization of a gas into an annular configuration.

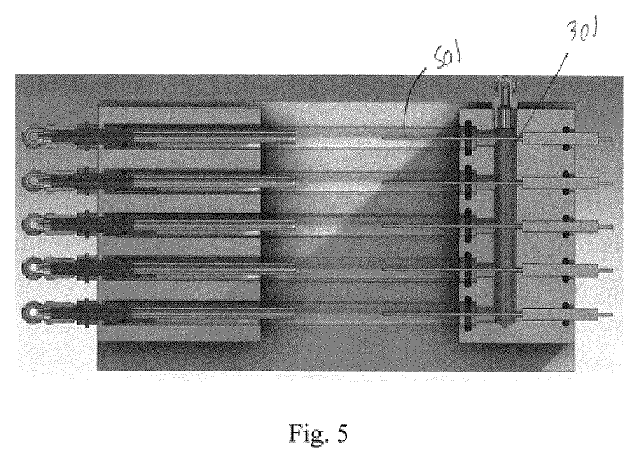

In one embodiment, air is the feedstock. Since air tends to be difficult to ionize, in one embodiment, a plasma starter is used. The plasma starter may vary according to application. In one embodiment, an easily-ionized gas, such as, as helium, is briefly introduced in the input gas to start the ionization of air. Those of skill in the art will appreciate other means of initiating/plasmatizing a gas. In another embodiment, rather than priming the plasma beam with an easily-ionized gas, an igniter can be used to initially ionize the gas. For example, referring to FIGS. 5-7, one embodiment is shown in which each of the reactors comprises an igniter 501. In this embodiment, the igniter is a known igniter such as those used to ignite electric grills. One of skill in the art will understand and appreciate, in light of this disclosure, that other igniters can be used.

Although the PDEB system provides for high selectivity due to its controlled radial power density as described above, it may be preferred, in certain applications, to use additional components to purify the output gas. For example, in one embodiment, scrubbers can be used to remove unwanted impurities such as NO2 from the desired NO product stream. Such scrubbers are well-known and tend to utilize alkali solutions/solids (e.g., potassium, sodium, calcium—hydroxides). Alternatively, or in addition to scrubbers, converters may be used to convert one form NxOx to another desired form. For example, in one embodiment, molybdenum converters or stainless converters are used to convert NO2 to NO. Still other means of purifying the output stream will be obvious to those of skill the art in light of this disclosure.

Having thus described a few particular embodiments of the invention, various alterations, modifications, and improvements will readily occur to those skilled in the art. Such alterations, modifications, and improvements as are made obvious by this disclosure are intended to be part of this description though not expressly stated herein, and are intended to be within the spirit and scope of the invention. Accordingly, the foregoing description is by way of example only, and not limiting. The invention is limited only as defined in the following claims and equivalents thereto.

What is claimed is:

1. A method of generating an output gas, comprising:
plasmatizing an input gas with RF power propagating from a tip of an electrode to form an annular plasma sheath constrained by a tube with said RF power propagating within said annular plasma sheath; and
forming said output gas as said annular plasma sheath propagates away from said tip of said electrode.

2. The method of claim 1, wherein said RF power propagating within said annular plasma sheath provides a consistent radial power density within said RF annular plasma sheath at a given axial position along said tube.

3. The method of claim 1, wherein said radial power density is consistent because said annular plasma sheath is sufficiently narrow that said RF power is radially consistent through said annular plasma sheath.

4. The method of claim 1, wherein, as said RF power propagates away from said tip of said electrode, said RF power drops to a point below said eV of one or more components of said input gas causing a combination of said one or more components to form said output gas.

5. The method of claim 1, wherein said input gas is air and said output gas is nitric oxide (NO).

6. The method of claim 1, wherein said input gas is comprises a gas for ionization and at least one additional reactant gas.

7. A gas generator system, comprising:
a radio frequency (RF) power source; and
at least one reactor having a first end and a second end, said reactor comprising at least,
a gas input at said first end for receiving an input gas from a gas source;
an elongated tube having an axis;
an electrode disposed at said first end with at least a portion of said electrode axially disposed within said tube, said electrode operatively connected to said RF power source, said electrode having a tip configured to emit said RF power such said RF power propagates axially along said tube, said electrode defining a channel for receiving said input gas from said gas input and for exhausting said input gas into said tube such that said input gas flows axially and laminarly along said tube;
a gas output in fluid communication with said tube to receive an output gas from said tube;
wherein said RF power from said electrode and said flow of said input gas along said tube are sufficient for said RF power to plasmatize said input gas to form an annular plasma sheath constrained by said tube with said RF power propagating within said sheath;
wherein said output gas forms from said annular plasma sheath as said annular plasma sheath propagates away from said tip of said electrode.

8. The system of claim 7, further comprising: said gas source for supplying said input gas;

9. The system of claim 7, wherein said RF power is greater than 30W

10. The system of claim 7, wherein said RF power is greater than 100W

11. The system of claim 7, wherein said RF power is sufficient to exceed the electronic volt of one or more components of said input gas.

12. The system of claim 7, wherein said RF power is sufficient to exceed the electronic volt of O2.

13. The system of claim 7, wherein said RF power source comprises an RF generator (not shown) to generate RF power, and an RF tuning system electrically connected to said RF power source to increase voltage of said RF power.

14. The system of claim 7, wherein said electrode has an orifice axially defined at said tip.

15. The system of claim 7, further comprising an igniter disposed in said tube to initiate plasmatization of said input gas.

16. The system of claim 7, wherein said at least one reactor comprises a plurality of reactors.

17. The system of claim 16, wherein said gas output of each of said plurality reactors are connected to an exhaust manifold.

18. The system of claim 7, wherein said tube comprises a polymeric material.

19. The system of claim 6, wherein said electrode comprises tungsten.

20. The system of claim 6, wherein said input gas is air and said output gas is nitric oxide (NO).
